(19) United States
(12) Patent Application Publication
(10) Pub. No.: US 2025/0099094 A1
(43) Pub. Date: Mar. 27, 2025

(54) LOCKING JOLLS RETRACTOR

(71) Applicant: KING FAISAL UNIVERSITY, AL-AHSA (SA)

(72) Inventor: ABDULRAHMAN SALEH ALMULHIM, AL-AHSA (SA)

(21) Appl. No.: 18/371,365

(22) Filed: Sep. 21, 2023

(51) Int. Cl. A61B 17/02; A61B 90/30

(52) U.S. Cl. CPC A61B 17/0206; A61B 90/30; A61B 2090/309

(57) ABSTRACT

A locking Jolls retractor includes a first arm having a first handle portion at a distal end, and a first self-retaining pincher at a proximal end. A second arm includes a second handle portion at a distal end, and a second self-retaining pincher at a proximal end. The first and second arms are pivotally joined together at a central pivot point. A toothed locking bar is mounted on the first arm. A release lever is mounted on the second arm and is pivotable between an engaged position with the toothed locking bar, such that the first arm and the second arm may be locked in a fixed angular position, and a disengaged position in which the first arm and second arm are freely movable about the central pivot point. First and second LEDs may be mounted on the first and second arms, respectively, adjacent to the central pivot point.

### BACKGROUND

### Field

The disclosure of the present patent application relates to surgical retractors, and particularly to a locking Jolls retractor.

### Description of Related Art

A Jolls retractor (also sometimes referred to as a “Joll retractor”, “Friedman retractor”, or “Joll-Friedman retractor”) is a specialized surgical instrument commonly used for difficult incision sites. The Jolls retractor is frequently used for thyroid as well as vaginal surgeries due to its unique design, which gives it a better approach in such anatomical areas. A Jolls retractor typically includes a pair of self-retaining pinchers held on opposed hinged shanks connected by a screw lock. While such a design is advantageous in that the screw lock allows the retractor to be compact and ergonomic in certain procedures in which space is limited, the disadvantage of using a screw lock is that it is a slow mechanism for opening and closing the gap defined by the opposed shanks. In particular, the screw lock is a slow means by which to adjust the self-retaining pinchers between their maximally distant and minimally distant positions from each other, since the screw lock requires repeated unwinding and winding to cause expansion and retraction between the shanks. Thus, a locking Jolls retractor solving the aforementioned problems is desired.

### SUMMARY

The locking Jolls retractor includes a first arm having a first handle portion at a distal end thereof, and a first self-retaining pincher at a proximal end thereof, and, similarly, a second arm having a second handle portion at a distal end thereof, and a second self-retaining pincher at a proximal end thereof. The first arm and the second arm are pivotally joined together at a central pivot point. A toothed locking bar is mounted on an end thereof to the first arm, and a release lever is pivotally mounted on the second arm, such that the release lever is selectively, adjustably and releasably engageable with the toothed locking bar.

The first handle portion and the second handle portion may include finger loops, and the first handle portion of the first arm may be straight from the finger loop thereof to the central pivot, with a remainder of the first arm being convexly curved. Similarly, the second handle portion of the second arm may be straight from the finger loop thereof to the central pivot, with a remainder of the second arm being convexly curved.

One or more light emitting diodes (LEDs) may be mounted to the first and/or second arms adjacent the central pivot. Additionally, the first self-retaining pincher may include a first pincher arm defined by the proximal end of the first arm and a first pivoting jaw member pivotally joined to the first arm. The first pivoting jaw member may have a first pivoting pincher arm at a proximal end thereof and a first holding surface defined at a distal end thereof. Similarly, the second self-retaining pincher may include a second pincher arm defined by the proximal end of the second arm and a second pivoting jaw member pivotally joined to the second arm. The second pivoting jaw member may have a second pivoting pincher arm at a proximal end thereof and a second holding surface defined at a distal end thereof. First and second clasps may be mounted to the first and second arms, respectively, for respectively selectively engaging and releasably locking the first and second holding surfaces.

Additionally, the first pivoting jaw member and the second pivoting jaw member may each be elastically biased. Similarly, the release lever may be elastically biased.

These and other features of the present subject matter will become readily apparent upon further review of the following specification.

### BRIEF DESCRIPTION OF DRAWINGS

FIG. 1 is a plan view of a locking Jolls retractor.

FIG. 2 is a plan view of the locking Jolls retractor, showing self-retaining pinchers thereof in locked and closed configuration.

FIG. 3 is a plan view of the locking Jolls retractor, showing relative movement between first and second arms thereof.

Similar reference characters denote corresponding features consistently throughout the attached drawings.

### DETAILED DESCRIPTION

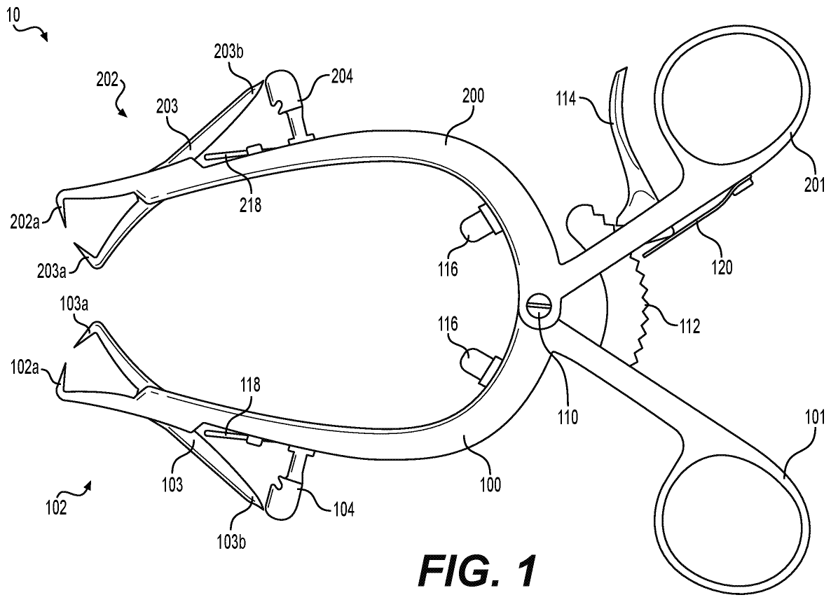

The locking Jolls retractor 1 is shown in FIG. 1 and includes first arm 100 having a first handle portion 101 at a distal end thereof, and a first self-retaining pincher 102 at a proximal end thereof. A second arm 200 includes a second handle portion 201 at a distal end thereof, and a second self-retaining pincher 202 at a proximal end thereof. The first and second arms 100, 200 are pivotally joined together at a central pivot point 110. It should be understood that any suitable type of pivotal connection may be used, such as a pivot pin or the like. A toothed locking bar 112 is mounted on one end to the handle portion 101 of first arm 100 and extends towards the second arm 200. As shown in FIG. 1, the opposite free end of the toothed locking bar 112 may be positioned beyond the handle portion 201. A release lever 114 is mounted on the handle portion 201 of the second arm 200 and is pivotable between an engaged position with the toothed locking bar 112, in which the first arm 100 and second arm 200 are in a fixed, locked angular position with respect to one another, and a disengaged position in which the first arm 100 and second arm 200 are freely movable with respect to one another, about the central pivot point 110.

In the non-limiting example of FIG. 1, the first handle portion 101 and the second handle portion 201 are shown as having finger loops. It should be understood that the overall configuration, size, shape and relative dimensions of the first and second handle portions 101, 201 are shown for exemplary purposes only. As shown, in this non-limiting example, the first handle portion 101 is substantially straight from the finger loop to the central pivot 110, with the remainder of the first arm 100 (beyond the central pivot 110) convexly curving (away from the second arm 200), terminating in a free end which defines a first pincher arm 102a. Similarly, the second handle portion 201 is substantially straight from its finger loop to central pivot 110, with the remainder of the second arm 200 (beyond the central pivot 110) convexly curving (away from the first arm 100), also terminating in a free end which defines a second pincher arm 202a.

As shown, the locking Jolls retractor 10 may also include at least one light emitting diode (LED). In the non-limiting example of FIG. 1, two such LEDs 116 are respectively mounted on interior sides of the first and second arms 100, 200, adjacent the central pivot 110. It should be understood that LEDs 116 may be powered by any suitable means, such as through connection with an integral battery or the like. The LEDs 116 are provided to illuminate the surgical area during a procedure and provide increased visibility.

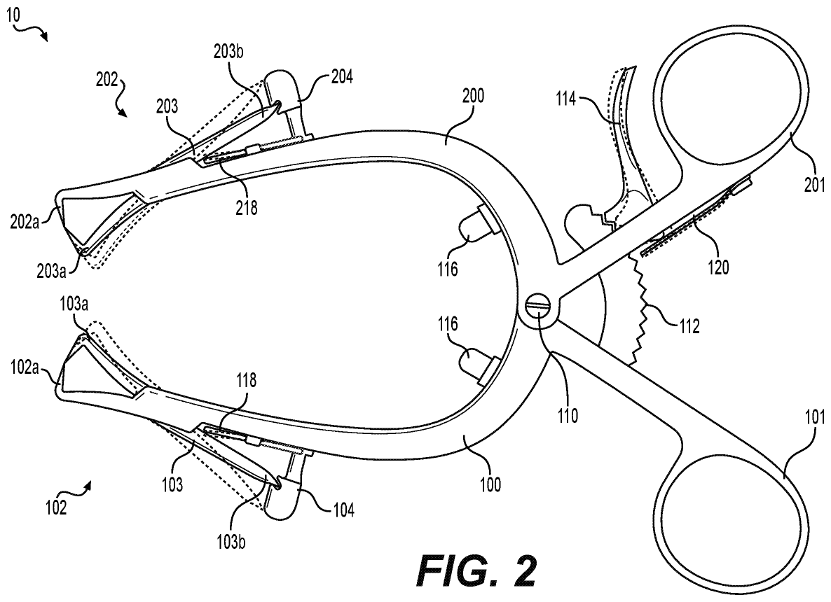

A first pivoting jaw member 103 is pivotally mounted on the first arm 100. The first pivoting jaw member 103 has a first pivoting pincher arm 103a at a proximal end thereof, and a first holding surface 103b at a distal end thereof. A first clasp 104 is mounted on the first arm 100 such that the first holding surface 103b can be moved between the free, disengaged position shown in FIG. 1 and the locked, engaged position of FIG. 2, where the first clasp 104 releasably locks the first pivoting pincher arm 103a in place. Similarly, a second pivoting jaw member 203 is pivotally mounted on the second arm 200. The second pivoting jaw member 203 has a second pivoting pincher arm 203a at a proximal end thereof, and a second holding surface 203b at a distal end thereof. A second clasp 204 is mounted on the second arm 200 such that the second holding surface 203b can be moved between the free, disengaged position shown in FIG. 1 and the locked, engaged position of FIG. 2, where the second clasp 204 releasably locks the second pivoting pincher arm 203a in place. It should be understood that first and second pivoting jaw members 203 and release lever 114 may each be spring-loaded by, for example, one or more leaf springs 118, 218, 120, respectively, or by any other suitable type of elastic biasing.

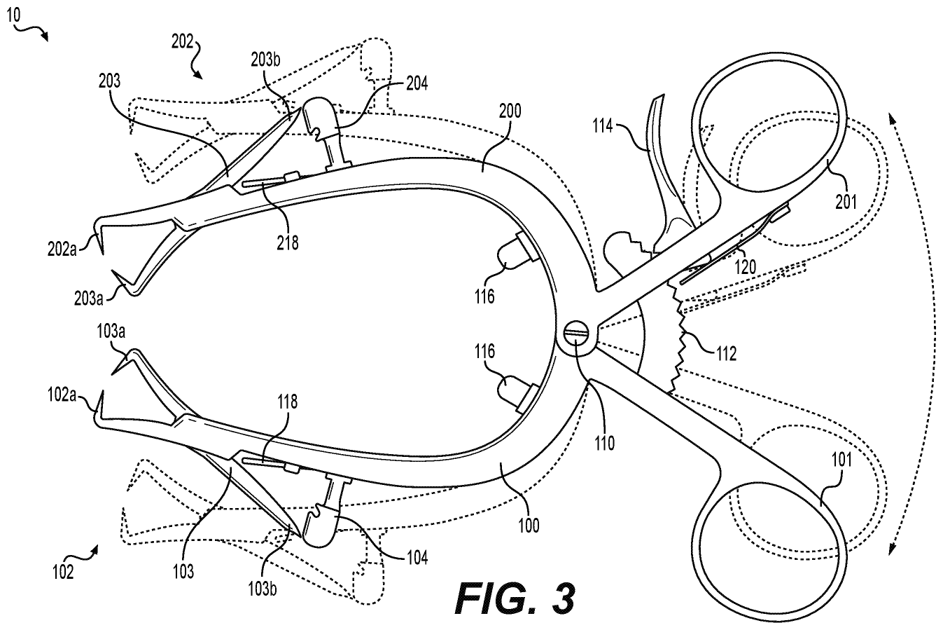

FIG. 3 illustrates the pivoting motion of first arm 100 and second arm 200 about central pivot point 110. With the addition of release lever 114 and toothed locking bar 112, arms 100 and 200 may be quickly adjusted between a fully closed and a fully open position, simply by disengaging lever 114 with toothed locking bar 112 and using the handle portions 101 and 201 to move arms 100 and 200 to the desired angular position.

It is to be understood that the locking Jolls retractor is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A locking retractor comprising:
a first arm having a first handle portion at a distal end thereof, and a first self-retaining pincher at a proximal end thereof, wherein the first self-retaining pincher comprises a first pincher arm defined by a proximal end of the first self-retaining pincher and a first pivoting jaw member pivotally joined to the first pincher arm, wherein the first pivoting jaw member has a first pivoting pincher arm at a proximal end thereof and a first holding surface defined at a distal end thereof;
a second arm having a second handle portion at a distal end thereof, and a second self-retaining pincher at a proximal end thereof, wherein the second self-retaining pincher comprises a second pincher arm defined by a proximal end of the second self-retaining pincher and a second pivoting jaw member pivotally joined to the second pincher arm, wherein the second pivoting jaw member has a second pivoting pincher arm at a proximal end thereof and a second holding surface defined at a distal end thereof, and wherein the first arm and the second arm are pivotally joined together at a central pivot point;
first and second clasps mounted to the first and second arms, respectively, for respectively selectively engaging and releasably locking the first and second holding surfaces, wherein said first and second clasps each have a recess for respectively selectively engaging and releasably locking the first and second holding surfaces defined at said distal ends thereof;
a toothed locking bar mounted on an end thereof to the first arm;
a first LED mounted to the first arm on an interior side of the first arm adjacent the central pivot;
second LED mounted to the second arm on an interior side of the second arm adjacent the central pivot; and
a release lever pivotally mounted on the second arm, wherein the release lever is selectively, adjustably and releasably engageable with the toothed locking bar and wherein the release lever is also selectively, adjustably, and releasably disengageable with the tooth locking bar.

2. The locking retractor as recited in claim 1, wherein the first handle portion and the second handle portion include finger loops.

3. The locking retractor as recited in claim 2, wherein the first handle portion of the first arm is straight from the finger loop thereof to the central pivot, a remainder of the first arm being convexly curved, and wherein the second handle portion of the second arm is straight from the finger loop thereof to the central pivot, a remainder of the second arm being convexly curved.

4-8. (canceled)

9. The locking retractor as recited in claim 1, wherein the first pivoting jaw member and the second pivoting jaw member are each elastically biased.

10. The locking retractor as recited in claim 1, wherein the release lever is elastically biased.
